(19) United States
(12) Patent Application Publication
(10) Pub. No.: US 2020/0352923 A1
(43) Pub. Date: Nov. 12, 2020

(54) TREATMENT OF SIDE EFFECTS OF BOTULINUM THERAPIES

(71) Applicant: DELNOVA, INC., Highland Park, IL (US)

(72) Inventor: Mary Gardner, Highland Park, IL (US)

(21) Appl. No.: 16/764,410

(22) PCT Filed: Nov. 15, 2018
(86) PCT No.: PCT/US2018/061337

§ 371 Date: May 15, 2020

Related U.S. Application Data
(60) Provisional application No. 62/586,362, filed on Nov. 15, 2017.

(51) Int. Cl. A61K 31/4406; A61K 31/16; A61K 9/00; A61P 13/10; A61P 13/02

(52) U.S. Cl. CPC A61K 31/4406; A61K 31/16; A61P 13/02; A61K 9/0053; A61P 13/10; A61K 9/0019

(57) ABSTRACT

The disclosure is directed to the use of a pharmaceutical product to accelerate recovery of adverse side-effects resulting from neurotoxin therapy for bladder dysfunction.

### CROSS-REFERENCE TO RELATED APPLICATIONS

This application claims the benefit of U.S. Provisional Patent Application No. 62/586,362, filed Nov. 15, 2017, which is incorporated herein by reference in their entireties.

### BACKGROUND OF THE DISCLOSURE

### 1. Field of the Disclosure

The disclosure is directed to the use of a pharmaceutical product to accelerate recovery of adverse side-effects resulting from neurotoxin therapy for bladder dysfunction.

### 2. Technical Background

Overactive bladder (OAB) also referred to as urinary urgency, is a treatable medical condition that afflicts approximately 16% of the U.S. population, both men and women. Symptoms include urinary frequency, urgency, nocturia (i.e., nighttime need to urinate), and accidental loss of urine (urge incontinence) due to a sudden need to urinate. Urge incontinence is usually associated with an overactive detrusor muscle.

Overactive bladder affects women more commonly than men (2.0%-19.0% vs 0.3%-8.9%). It is also more prevalent amongst older patients, e.g., after the age of 44 in women and after 64 in men. There are numerous OAB costs, and those include costs related to pad use, pharmacotherapy, catheters, physician time, outpatient and inpatient visits, as well as loss of productivity. One study in Germany found the cost of OAB to be almost 3.57 billion Euros per year.

But the costs are not the only concern associated with OAB. There are also further health consequences that include a higher incidence of urinary tract infections (UTIs) which may be accompanied by a higher risk of kidney stones and possibly even sepsis. OAB also adversely affects the quality of life, quality of sleep, productivity, and mental health of patients. Associated comorbidities and complications include falls, adult diaper rash, and depression or anxiety. One burden-of-illness model estimated that the annual cost of burden of OAB is $65.9 billion.

OAB is a challenging condition with many causes or mimics of overactive bladder symptoms. Such causes or mimics include neurologic injuries (e.g., spinal cord injury or stroke), neurologic diseases (e.g., multiple sclerosis, dementia, Parkinson's disease, medullary lesions, diabetic neuropathy, etc.), infection (e.g., urinary tract infection or interstitial cystitis), cardiology conditions (e.g., congestive heart failure or use of diuretics), and many others (e.g., bladder calculi, stool impaction, diabetes, bladder cancer or carcinoma in situ, etc.)

Because OAB is a challenging condition, it is not readily treated using oral medications. Botulinum toxin Type A or onabotulinumtoxinA, approved by the U.S. Food and Drug Administration and available under various brand names including BOTOX® (Allergan, Irvine, Calif., USA; herein “BOTOX”), has become an accepted and approved treatment for the condition of bladder dysfunction and more particularly overactive bladder(OAB). In practice, the therapy is used in a subset of patients described as refractory overactive bladder (ROAB). In addition, several clinical trials are currently in progress to further evaluate BOTOX formulations or novel drug delivery regimens in treatment of OAB: Clinical Trial No. NCT00583219 sponsored by Mayo Clinic for delivery by intravesical instillation (botulinum toxinA, DMSO); No. NCT03052764 sponsored by Allergan for 2 injections, 1 week apart; No. NCT03385460 sponsored by Allergan for intravesical instillation with shock waves; No. NCT03320850 sponsored by Allergan for intravesical instillation with admixture with RTGel™; No. NCT01167257 sponsored by Buddhist Tzu Chi General Hospital for intravesical instillation with lipotoxin (liposome encapsulated BOTOX); No. NCT02735499 sponsored by the Hospital of Vestfold, Norway for intravesical instillation with electromotive drug application (EMDA); and No. NCT02674269 sponsored by Urogen Pharma Ltd. For intravesical instillation with BotuGel™ and/or RTGEL™ BOTOX. One clinical trial is currently evaluating the use in interstitial cystitis: Clinical Trial No. NCT01997983 sponsored by Urogen Pharma Ltd. for delivery by intravesical instillation using 3TC-Ge).

In clinical practice, treatment generally starts with behavioral or physical interventions, if these prove unsuccessful then pharmacological treatments are prescribed (anticholinergics and the newer beta-three agonists). This accounts for about a 20% subset of OAB patients. These drugs are often not effective, and after 2 years there is a significant rate of non-compliance with only 6-12% of patients that continue their medication. There are also undesirable side effects such as dry-mouth and constipation. Patients that are non-responsive to first-line pharmacological therapies are considered to have “refractory overactive bladder”. Second line of treatment includes sacral neurostimulation with an implantable device, percutaneous posterior tibial nerve stimulation (multiple weekly visits) and oncobotulinumA (e.g., BOTOX injections into the bladder). The recommended dose for treatment of ROAB with BOTOX is 100 Units of BOTOX. The recommended dilution is 100 Units/10 mL with preservative-free 0.9% Sodium Chloride Injection, USP. The effect of BOTOX treatment also diminishes with time, and patients can be considered for re-injection. Median time to qualify for re-treatment in the double-blind, placebo-controlled clinical studies was 295-337 days (e.g., 42-48 weeks) for 200 Units of BOTOX, but no sooner than 12 weeks from the prior bladder injection.

In the treatment of refractory OAB, reconstituted BOTOX (100 Units/10 mL) is injected into the detrusor muscle (intradetrusor injection, see FIG. 1) via a flexible or rigid cystoscope, from within the bladder lumen, usually at 20-30 sites across the dome of the bladder. But prior to treatment, the physician administers an anesthetic to numb the bladder, and bladder is instilled with enough saline to achieve adequate visualization for the injections. The recommended dose for neurogenic OAB (cause is from brain, spinal cord, or nerve condition) is 200 Units.

BOTOX leads to lax paralysis, because the acetylcholine from cholinergic motor-nerve endings is not released to excite the muscle. Acetylcholine is considered to be responsible for detrusor contractions. When injected into the detrusor muscle, BOTOX is taken up by the presynaptic nerve terminal via endocytosis, binds to the SNARE protein complex, and prevents the binding and subsequent release of acetylcholine from the presynaptic nerve terminal. This prevents stimulation of muscarinic receptors in the bladder detrusor muscle. Thus, the treatment with BOTOX is not without adverse effects.

Although BOTOX was found to have better efficacy vs. the implant in head to head clinical trial published in 2016, BOTOX had a greater risk of urinary tract infections. 35% of women receiving BOTOX had incidence of urinary tract infections compared to 11% of women with the implant.

In addition, Allergan clinical trials showed a 6% incidence of urinary retention amongst the 552 patients treated with BOTOX. These patients were temporarily unable to fully empty their bladders on their own after treatment and required self-catheterization to empty the bladder until the bladder can be emptied on its own. In patients affected by a neurologic condition, the rate of self-catheterization was as high as 30.6% of patients following treatment with BOTOX 200 Units as compared to 6.7% of patients (7/104) treated with placebo. There are other reports that also suggest that voiding difficulty is experienced in about 10% of patients.

Onset of urinary retention typically coincides with the beginning of efficacy 5 -10 days after injection. The duration of retention is variable, with some patients only requiring catheterization for a few days, while for others the condition persists for the duration of the effects of BOTOX. A group of women receiving 100 Units of BOTOX was monitored for urinary retention, and 5% of women remained in retention at 2 months, 3% remained in retention at 4 months, and 1% remained in retention at 6 months.

Urinary retention is managed by physical bladder drainage using a catheter, such as using clean intermittent self-catheterization (CISC). CISC is now considered the gold standard for the management of urinary retention. Failure to empty the bladder may expose the patient to significant complications, such as urinary urgency, frequency, nocturia, incontinence, recurrent urinary tract infections (UTIs), bladder stones, upper urinary tract changes, and even renal impairment. Nevertheless, there is significant reluctance by patients to initiate CISC. Internal barriers range from physical and psychological factors, to the understanding of the importance, the patient's perception of the treatment, and its implications. External factors that may influence adherence include quality of the teaching, supervision, reassurance, and follow-up. As a result, patients must be able and willing to perform, or accept the possibility of, CISC if BOTOX injections are planned to be offered.

Generally, the most frequently reported adverse reactions for OAB occurring within 12 weeks of BOTOX injection include urinary tract infection (18% vs. 6% for placebo), dysuria (9% vs. 7% for placebo), urinary retention (6% vs. 0% for placebo), bacteriuria (4% vs. 2% for placebo), and residual urine volume (3% vs.0% for placebo). For OAB associated with a neurologic condition, the most frequently reported adverse reactions within 12 weeks of BOTOX injection include urinary tract infection (24% vs. 17% for placebo), urinary retention (17% vs. 3% for placebo), and hematuria (4% vs. 3% for placebo).

Due to side effects associated with BOTOX treatment, according to web reviews, patient satisfaction is marginal at best with less than 6 in 10 patients being satisfied. The most patient dissatisfaction is centered on an inability to urinate and complete reliance on catheters, sometimes for years following a single treatment. As a result, there remains a need to minimize the side effects and increase patient satisfaction when receiving BOTOX therapy for OAB.

### SUMMARY OF THE DISCLOSURE

The present inventor has found that local administration of a rescue agent can be used to accelerate recovery from side effects. In addition, a peripherally acting neurotoxin does not cross the blood brain barrier and as such offers inherently less risk to the patient. Local administration of the antcholinesterase, further limits the potential for side-effects resulting from the rescue agent. Specifically, the present inventor found that locally delivering an anticholinesterases reversal agent is effective for mitigating urinary retention following treatment with botulinum toxin for managing symptoms of overactive bladder.

Thus, in one aspect, the disclosure provides methods of mitigating the side-effects of botulinum toxin therapy in a patient in need thereof. In general, the patient has received botulin toxin for treating overactive bladder or urinary urgency. Such methods include locally administering one of more of anticholinesterases to a bladder muscle denervated by a botulinum toxin.

In certain embodiments of the methods of the disclosure as described herein, the one or more anticholinesterases includes, but is not limited to, neostigmine, edrophonium, pyridostigmine, physostigmine, rivastigmine, and combinations thereof. In certain embodiments of the methods of the disclosure as described herein, the one or more anticholinesterases is pyridostigmine. In certain embodiments of the methods of the disclosure as described herein, the one or more anticholinesterases is rivastigmine.

In another aspect, the disclosure provides use of one or more anticholinesterases to mitigate the side-effects of botulinum toxin therapy in a patient of the disclosure as described herein. The one or more anticholinesterases is locally administered to a bladder muscle denervated by a botulinum toxin.

In certain embodiments of the uses of the disclosure as described herein, the one or more anticholinesterases includes, but is not limited to, neostigmine, edrophonium, pyridostigmine, physostigmine, rivastigmine, and combinations thereof. In certain embodiments of the uses of the disclosure as described herein, the one or more anticholinesterases is pyridostigmine. In certain embodiments of the methods of the disclosure as described herein, the one or more anticholinesterases is rivastigmine.

### BRIEF DESCRIPTION OF THE DRAWINGS

The accompanying drawings are included to provide a further understanding of the methods and compositions of the disclosure, and are incorporated in and constitute a part of this specification. The drawings illustrate one or more embodiment(s) of the disclosure and, together with the description, serve to explain the principles and operation of the disclosure.

FIG. 1 illustrates the injection pattern for intradetrusor injections for treatment of overactive bladder and detrusor overactivity associated with a neurologic condition provided in the product insert for BOTOX for injection (Allergan, Irvine, Calif., USA).

FIG. 2 illustrates the anatomy of the bladder wall.

FIG. 3A illustrates recovery from BOTOX inhibition of force production in bladder strips by pyridostigmine. Data presents average±SEM over time normalized to force produced at 100^{th} minute. Strips excised from a rodent were exposed to 4 Units of BOTOX at 100 minutes and incubated for 2 hours. Another set of strips were not exposed to BOTOX. Recording was restarted, and force drop was measured for an additional 90 minutes, at which point strips were exposed to 0.5 mM of pyridostigmine (Pyr, n=4) or vehicle (Vehicle, n=3). FIG. 3B presents an average±SEM of force produced in each group at t=100 (Baseline), t=315 minutes (Phase I, post-BOTOX) and t=425 minutes (Phase II, post-Pyr, recovery). FIG. 3C is a representative force trace from a bladder strip. Trace is a 3-min recording, starting with a 5-second electrical field stimulation (EFS).

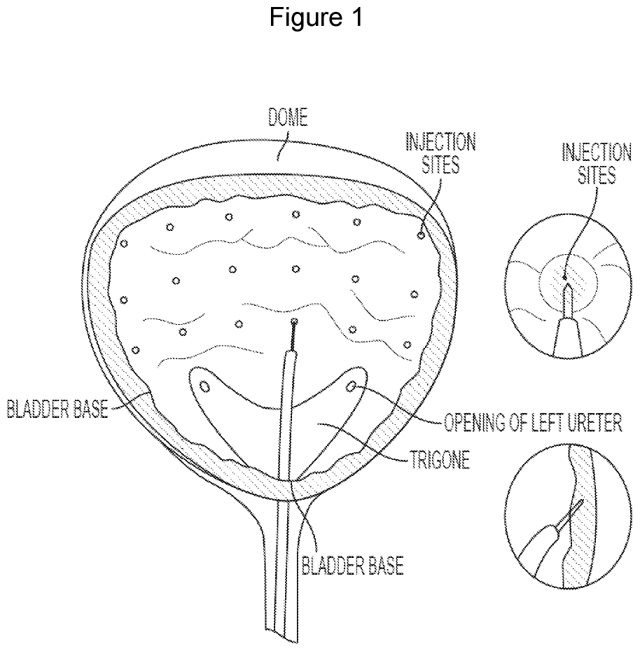

### DESCRIPTION OF THE DISCLOSURE

Before the disclosed methods and materials are described, it is to be understood that the aspects described herein are not limited to specific embodiments, apparatus, or configurations, and as such can, of course, vary. It is also to be understood that the terminology used herein is for the purpose of describing particular aspects only and, unless specifically defined herein, is not intended to be limiting.

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements or steps but not the exclusion of any other integer or step or group of integers or steps.

As used in the specification and the appended claims, the singular forms “a,” “an” and “the” include plural referents unless the context clearly dictates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

An “effective amount” refers to that amount of a compound which, when administered to a subject, is sufficient to effect treatment for condition described herein. The amount of a compound which constitutes an “effective amount” will vary depending on the compound, the disorder and its severity, and the age of the subject to be treated, but can be determined routinely by one of ordinary skill in the art.

The term “denervation,” “chemical denervation,” or “chemodenervation” as used herein means loss of nerve supply (i.e., block of neural transmission) caused by an agent (e.g., chemical compound).

The term “non-invasive” refers to a medical procedure which does not require break in the tissue or bladder wall (e.g., no injection or incision) or removal of tissue. In general, non-invasive procedure causes no injury to tissue. In contrast, a needle injection is an invasive medical procedure due to its use as a puncturing device.

The terms “intravesical instillation” refers to a procedure of exposing the bladder to the desired solution containing the medicament and filling the bladder via a catheter without puncturing the bladder muscle by injection.

In view of the present disclosure, the methods and active materials described herein can be configured by the person of ordinary skill in the art to meet the desired need. In general, the disclosed methods and materials provide targeted, localized parenteral or intravesical administration of an anticholinesterase to the affected patient to mitigate the side-effects of botulinum toxin therapy, wherein the patient has received botulin toxin for treating overactive bladder or urinary urgency.

For example, in some embodiments, the methods of the disclosure address the unwanted side effects associated with BOTOX use, such as those resulting from an excess response to the muscle blocker. In general, currently there is no remedy to safely mitigate the unintended adverse effects of BOTOX—the only option is to wait until the effect of the drug subsides, sometimes weeks to months. The inventors have found that anticholinesterases, generally not recommended for use in non-life threatening situations due to their own side effects, are useful in mitigating the adverse effects of BOTOX. Accordingly, the disclosure is directed to target administration of an anticholinesterase. Specifically, the inventors have found that the anticholinesterases can be used in the methods of the disclosure by targeted administration and/or in minimal dose by administering only to the afflicted tissue.

In certain embodiments, the local injection (e.g., to the bladder wall) of the one or more anticholinesterases can be useful in the methods of the disclosure.

In certain embodiments, the formulations of the one or more anticholinesterases are administered by intravesical instillation. For example, in certain embodiments, a formulation suitable for intravesical instillation includes the one or more anticholinesterase and one or more mucosal adherents and/or one or more penetration enhancers. In other embodiments, the one or more anticholinesterase can be formulated into a sustained release formulation suitable for intravesical instillation. In other embodiments, the one or more anticholinesterase can be formulated into a formulation suitable for delivery by a medical device capable of sustained release.

The neurotoxin botulinum is used in a broad range of cosmetic and medical procedures. In the treatment of OAB, there are numerous undesirable side effects. The neurotoxin acts to block the release of ACh, at the neuromuscular junction. The anticholinesterase acts to indirectly increase ACh by degrading the endogenous AChE. The binding of ACh to its receptor sites is necessary to maintain muscle transmission. The depth of block is a critical factor which dictates the efficacy of the anticholinesterase in accelerating spontaneous recovery. In order for recovery or muscle reactivation to occur, ACh needs to be present. As used herein, the term “depth of the block” refers to the level of occupancy of postsynaptic receptors. Due to the nature of paralysis and spread induced by botulinum toxin in commercial use, partial chemical denervation and/or natural recovery will occur. Therefore, the condition or depth of block will determine the speed of recovery. The present inventor has found that peripherally acting anticholinesterase can be used as a neurotoxin rescue agent accelerating the time to recovery. The inventor has found that the timing of dosing of anticholinesterase, the concentration of anticholinesterase, and period of dosing are important elements in the efficacy of the rescue treatment.

Thus, in one aspect, the disclosure provides methods of mitigating the side-effects of botulinum toxin therapy in a patient in need thereof, wherein the patient has received botulin toxin for treating overactive bladder or urinary urgency. Such methods include locally administering one of more of anticholinesterases to a bladder muscle denervated by a botulinum toxin.

The botulinum toxin is a neurotoxic protein produced by Clostridium botulinum and related species. Strains of Clostridium botulinum produce seven distinct neurotoxins designated as types A-G. All seven types have a similar structure and molecular weight, consisting of a heavy (H) chain and a light (L) chain joined by a disulfide bond and they all interfere with neural transmission by blocking the release of acetylcholine. Therefore, in one embodiment, the botulinum toxin of the disclosure includes one or more of Type A, Type B, Type C, Type D, Type E, Type F, and Type G. In one embodiment, the botulinum toxin of the disclosure includes one or more of Type A, Type B, Type E, and Type F. In one embodiment, the botulinum toxin of the disclosure includes one or more of Type A and Type B. In one embodiment, the botulinum toxin of the disclosure is botulinum toxin Type A (also known as onabotulinumtoxinA, incobotulinum toxin A, abobotulinum toxin A, BOTOX®, or DYSPORT®). As the mechanism of action of botulinum toxin Type A products is similar, one skilled in the art recognizes that other comparable botulinum neurotoxins may be used.

The methods of the disclosure require a composition comprising an anticholinesterase. Anticholinesterases (i.e., cholinesterase inhibitors) fall into two classes, organophosphorus compounds, which are non-reversible, and carbamates, which are reversible. The former generally have higher toxicity, longer duration of action, and are often associated with central nervous system (CNS) toxicity. Reversible anticholinesterases have found applications in medicine for a broad range of indications. For example, some reversible anticholinesterases are used in treatment of Alzheimer's disease as these can cross the blood brain barrier to reach the CNS.

In some embodiments, the anticholinesterase of the disclosure is a reversible anticholinesterase. In some embodiments, the anticholinesterase of the disclosure is a reversible anticholinesterase having one or more of groups selected from carbamate, tertiary ammonium, and quaternary ammonium.

The selection of anticholinesterase will also depend upon the method of administration (e.g., targeted administration) and the specific anticholinesterase attributes. Targeted administration is covered in greater detail below.

In some embodiments, the anticholinesterase is selected from one or more of: physostigmine, neostigmine, ambenonium, pyridostigmine, ambenonium, demecarium, rivastigmine, galantamine, donepezil, tacrine, 7-methoxytacrine, edrophonium, huperzine A, ladostigil, and any derivative and combinations thereof.

In some embodiments, the anticholinesterase of the disclosure is selected from one or more of:

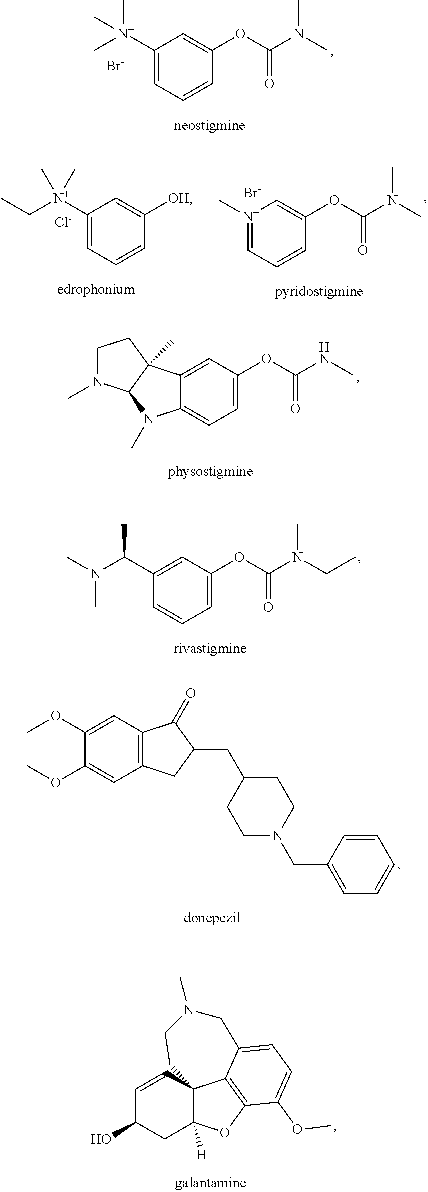

and a combination thereof.

In certain embodiments of the disclosure, the anticholinesterase is neostigmine, edrophonium, pyridostigmine, physostigmine, rivastigmine, and combinations thereof. In some embodiments of the disclosure, the anticholinesterase is pyridostigmine, neostigmine, edrophonium, rivastigmine, or a combination thereof.

In some embodiments of the disclosure, the anticholinesterase is pyridostigmine. Pyridostigmine is not lipid soluble and as such is peripherally acting. This property makes it desirable for use in muscle related conditions. Pyridostigmine is also safer as compared to neostigmine due to fewer incidences of bradycardia and arrhythmias.

In certain embodiments of the methods of the disclosure as described herein, the one or more anticholinesterases is rivastigmine. Rivastigamine is lipid soluble and has a greater affinity to pass through protein barriers such as skin or bladder wall.

In some embodiments of the disclosure, the anticholinesterase is a compound of formula (I):

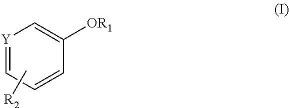

or a pharmaceutically acceptable salt thereof, wherein
Y is CR³ or N⁺X⁻R⁴, wherein X is a halogen;
R₁ is selected from hydrogen, C₁-C₆ alkyl, —CO(OH), —CO(C₁-C₆ alkoxy), —CO(NH₂), —CONH(C₁-C₆ alkyl), and —CON(C₁-C₆ alkyl)₂;
R₂ is hydrogen, or R₂ and R₃ together with the atoms to which they are attached form an optionally substituted heterocycle;
R₃ is selected from C₁-C₆ alkyl, C₁-C₆ alkoxy, hydroxy C₁-C₆ alkyl, amino C₁-C₆ alkyl, (C₁-C₆ alkylamino) C₁-C₆ alkyl, (di C₁-C₆ alkylamino) C₁-C₆ alkyl, C₁-C₆ alkoxy C₁-C₆ alkyl —OH, —NH₂, —NH(C₁-C₆ alkyl), —N(C₁-C₆ alkyl)₂, and —N⁴(C₁-C₆ alkyl)₃X⁻; and
R₄ is selected from C₁-C₆ alkyl, hydroxy C₁-C₆ alkyl, or C₁-C₆ alkoxy C₁-C₆ alkyl.

In some embodiments, the compound of formula (I) is wherein Y is C. In some embodiments, the compound of formula (I) is wherein Y is or N⁺X⁻, or Y is N⁺Br⁻ or N⁺Cl⁻, or Y is N⁺Br⁻.

In some embodiments, the compound of formula (I) according to any one of preceding embodiments is wherein R₁ is selected from hydrogen, C₁-C₆ alkyl, —CO(NH₂), —CONH(C₁-C₆ alkyl), and —CON(C₁-C₆ alkyl)₂. In some embodiments, the compound of formula (I) is wherein R₁ is selected from hydrogen, —CO(NH₂), —CONH(C₁-C₆ alkyl), and —CON(C₁C₆ alkyl)₂. In some embodiments, the compound of formula (I) is wherein R₁ is hydrogen. In some embodiments, the compound of formula (I) is wherein R₁ is —CO(NH₂), —CONH(C₁-C₆ alkyl), or —CON(C₁-C₆ alkyl)₂. In some embodiments, the compound of formula (I) is wherein R₁ is —CON(C₁-C₆ alkyl)₂.

In some embodiments, the compound of formula (I) according to any one of preceding embodiments is wherein R₂ is hydrogen. In some embodiments, the compound of formula (I) according to any one of preceding embodiments is wherein R₂ together with R₃ and the atoms to which they are attached form an optionally substituted heterocycle. In some embodiments, the heterocycle is optionally substituted with one or more of halogen, C₁-C₆ alkyl, C₁-C₆ haloalkyl, C₁-C₆ alkoxy, —OH, —NH₂, —NH(C₁-C₆ alkyl), or —N(C₁-C₆ alkyl)₂. In some embodiments, the heterocycle is octahydropyrrolo[2,3-b]pyrrole or pyrrolidine, each optionally substituted with one or more of halogen, C₁-C₆ alkyl, C₁-C₆ haloalkyl, C₁-C₆ alkoxy, —OH, —NH₂, —NH(C₁-C₆ alkyl), or —N(C₁-C₆ alkyl)₂. In some embodiments, the heterocycle is octahydropyrrolo[2,3-b]pyrrole optionally substituted with one or more of halogen, C₁-C₆ alkyl, C₁-C₆ haloalkyl, C₁-C₆ alkoxy, —OH, —NH₂, —NH(C₁-C₆ alkyl), or —N(C₁-C₆ alkyl)₂.

In some embodiments, the compound of formula (I) according to any one of preceding embodiments is wherein R₃ is selected from C₁-C₆ alkoxy, —OH, —NH₂, —NH(C₁-C₆ alkyl), —N(C₁-C₆ alkyl)₂, and —N⁺(C₁-C₆ alkyl)₃X⁻. In some embodiments, R₃ is selected from C1-C₆ alkoxy and —OH. In some embodiments, R₃ is selected from —NH₂, —NH(C₁-C₆ alkyl), —N(C₁-C₆ alkyl)₂, and —N⁺(C₁-C₆ alkyl)₃X⁻. In some embodiments, R₃ is —N⁴(C₁-C₆ alkyl)₃X⁻. In some embodiments, R₃ is —N⁺(C₁-C₆ alkyl)₃Br⁻ or —N⁺(C₁-C₆ alkyl)₃C⁻.

In certain embodiments, the methods of the disclosure include targeted administration of the composition. Targeted administration to the site of action can enhance anticholinesterase efficacy. By increasing local active anticholinesterase concentration to the afflicted tissue, while minimizing exposure to other areas of the body, any anticholinesterase toxicity can be reduced. Localized administration avoids hepatic first-pass metabolism and gastrointestinal tract side effects associated with some anticholinesterases. Furthermore, the total anticholinesterase dose can be significantly lower, thereby reducing patient exposure and off-target systemic side effects. In certain embodiments, the methods of the disclosure allow widespread use of the anticholinesterase class of drugs (such as those only indicated for use in life threatening conditions). In one embodiment of the disclosure, the composition is administered directly to the muscle affected.

Currently, the most common method of administration to the bladder is oral administration. Other methods include local administration. Local administration of an active agent to the bladder may be achieved using, for example, intradetrusor injections or intravesical instillation. Intradetrusor injections are injections into the bladder wall or bladder muscle.

Intravesical instillation is delivery of a solution into the bladder, e.g., a method of topical delivery of active agents to the bladder without injection. Such solution or formulation for instillation may be optimized using an agent that will promote transmucosal delivery. Intravesical instillation is non-invasive as compared to intradetrusor injections. In recent years, greater attention has been given to the administration of the active agents directly to the bladder without injections. Such administration may avoid pain, hematuria, large post-void residual or urinary tract infection.

In certain embodiment of the methods of the disclosure, the administration is by intravesical instillation. Such administration, in certain embodiments, extends the dwell time of the one or more anticholinesterases within the bladder. In certain embodiments, the dwell time is extended by means of a medical device. In certain embodiments, the dwell time is extended by means of is formulating or co-administering the one or more anticholinesterase with an agent. Thus, in some embodiments, the one or more anticholinesterase is formulated with an agent to promote penetration into the bladder wall. In some embodiments, the one or more anticholinesterase is co-administered with an agent to promote penetration into the bladder wall. In some embodiments, the agent may be a mucoadhesive agent. Without being bound to a theory, it is believed that mucoadhesive agent serves to increase dwell time or contact between the anticholinesterase and bladder wall.

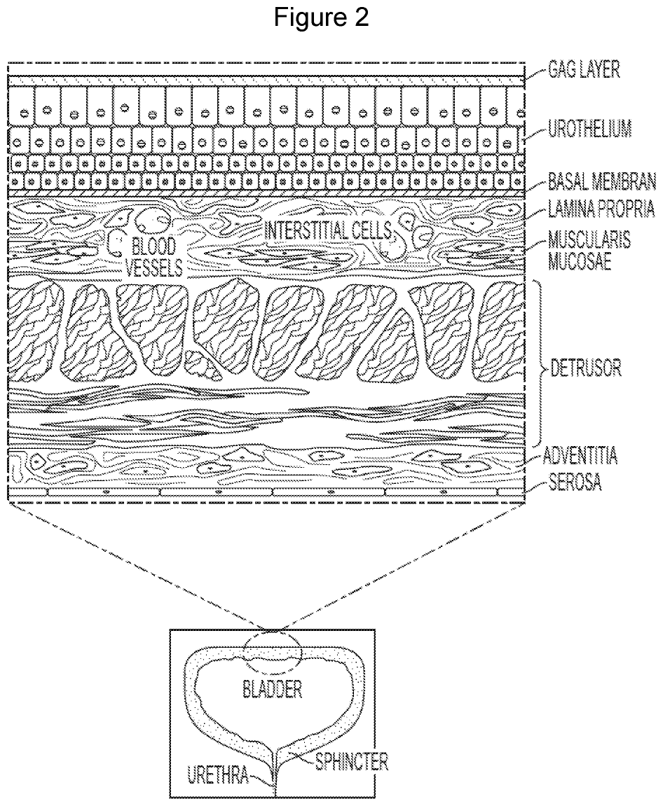

The bladder is a hollow smooth muscle pelvic organ whose function is the storage and clearance of urine. The anatomy of the bladder wall is shown in FIG. 2. The bladder wall has defined layers consisting of the innermost portion called the mucosa, the intermediate muscularis propria layer (detrusor muscle), and the outer adventitia/serosa layer. The impermeability is associated with the epithileum or ureithelium, as well as the glycasoaminoglycans (GAGs). As a result, the administration to the bladder is challenged by physical features pertaining to the bladder wall, as well as expansion and contraction of the bladder. This elastic organ fills with urine (e.g., 500 mL normal capacity). Thus, the active agents can be washed out because of intermixing, dilution, and voiding with urine.

Various approaches to administration of active agents may be employed to overcome the bladder permeability barrier. Strategies to overcome the barrier challenge include modification of the drug itself. For example, the anticholinesterase rivastigmine is lipophilic and is expected to be more successfully cross membrane barriers. The basic structure of the anticholinesterase may be modified to alter molecular charge, or include penetration enhancing formulations or binding to penetration enhancing molecules. Some approaches, such as but not limited to formulations containing mucoadhesives, increase the dwell time within the bladder.

One example of a mucoadhesive is pectin. Pectin acts to form a mucosal binding gel (i.e., mucoadhesive), Pectin is a natural, non-toxic and non-irritating ingredient, and it has been used in administration of intranasal fentanyl.

Reverse thermosensitive hydrogels, such are RTgel™, are under clinical development. Their purpose is to increase the dwell time of intravesical drugs by solidifying into a gel at body temperature. Similarly, mucoadhesive carriers increase dwell time by attaching to bladder epithelium. For example, chitosan is being evaluated for this purpose the main agent currently being investigated. Chitosan is a nontoxic, biodegradable, naturally occurring polysaccharide. It is thought that positive charge chitosan adheres to negative charge epithelial membrane thus rearranging cellular junctions, to promote permeability. Polymeric hydrogels, such as PEG-PLGA-PEG temperature sensitive polymer, may be used as in-situ gelling systems.

Penetration enhancers temporarily disrupt the tight packing of the urothelium; examples include chitosan, hyaluronan-phosphatidylethanolamine (HA-PE), dimethyl sulfoxide (DMSO), protamine sulfate, alcohol, benzalkonium chloride, surfactants (such as sodium lauryl sulfate, Tween 80®, etc.). Nanocarriers such as liposomes, gelatin nanoparticles, polymeric nanoparticles and magnetic particles may also be used to enhance transport.

In one embodiment of the methods of the disclosure, the one or more anticholinesterases is formulated with one or more permeation enhancers including surfactants, such as Poloxamer 407, Poloxamer 407 also known by the trademark Pluronic® F127, is a water-soluble, non-ionic triblock copolymer that is made up of a hydrophobic residue of polyoxypropylene (POP) between the two hydrophilic units of polyoxyethylene (POE).

To increase the dwell time of intravesical drugs, an intravesical anticholinesterase delivery device that is implanted in the bladder and left in place for an extended period of time can be used. This increases the amount of time the bladder mucosa is exposed to the anticholinesterase. One such device under development by Taris BioMedical is an implanted a micro osmotic infusion pump capable of releasing a known amount of drug over time. This device is inserted through the urethra by a Foley catheter into the bladder and removed post-therapy in-office by cystoscopy.

Those skilled in the art will recognize that the concentration or dose of the one or more anticholinesterases formulated for delivery to the bladder needs to be greater than the formulation or dose implemented for direct injection. Diffusion is controlled by a driving force towards a state of equilibrium where drug flux partially controlled by concentration gradient from high to low. In certain embodiments, the absolute anticholinesterase dose/concentration in an intravesical instillation may be 10 to 100 times higher than the anticholinesterase dose/concentration for intradetrusor injection. In certain embodiments, instillation volumes are in the range of about 10 mL to about 60 mL. In certain embodiments, dwell times are in the range of 2 seconds to 20 min.

### Mechanism of Action

There are seven serotypes of botulinum toxin (A-G) with botulinum toxin type A being the only FDA approved product to treat lower urinary tract symptoms. There are a few proprietary preparations of the neurotoxin botulinum toxin A. The two most studied preparations are onabotulinumtoxinA (BOTOX®, Allergan, Inc., Irvine, Calif., USA) and abobotulinumtoxinA (DYSPORT®, Ipsen Biopharm Ltd, Slough, UK).

BOTOX is an acetylcholine release inhibitor and a neuromuscular blocking agent. BOTOX is composed of a heavy and a light chain. The heavy chain selectively binds the toxin to the presynaptic cholinergic nerve terminal, while the light chain prevents acetylcholine vesicle release. With time, sprouting of new nerve terminals occurs and eventually the original endplate regains function. The inhibition of muscular contraction is therefore temporary. BOTOX blocks neuromuscular transmission by binding to acceptor sites on motor or autonomic nerve terminals, entering the nerve terminals, and inhibiting the release of acetylcholine. This inhibition occurs as the neurotoxin cleaves SNAP-25, a protein integral to the successful docking and release of acetylcholine from vesicles situated within nerve endings. When injected intramuscularly at therapeutic doses, BOTOX produces partial chemical denervation of the muscle resulting in a localized reduction in muscle activity. In addition, the muscle may atrophy, axonal sprouting may occur, and extrajunctional acetylcholine receptors may develop. There is evidence that reinnervation of the muscle may occur, thus slowly reversing muscle denervation produced by BOTOX. Following intradetrusor injection, BOTOX® affects the efferent pathways of detrusor activity via inhibition of acetylcholine release.

The neurotransmitter that is primarily responsible for communication between peripheral nerves and muscle cells is acetylcholine (ACh). This process enables muscle contraction. Neuromuscular blocking agents also compete with ACh. When ACh binding is inhibited, muscle contraction is blocked. There is also early evidence that BOTOX can reduce afferent sensitization by inhibiting neuropeptide release and decreasing firing frequency. This may contribute to its efficacy in urinary urgency (Youko et al. (2012) European Neurology 62:1157-1164).

Neuromuscular blockers have been used in surgery to generate a controlled, temporary state of paralysis. Generally these drugs are poorly orally absorbed, have poor lipid solubility and are administered parenterally. They are considered “high alert”medications when used in anesthesia. During surgery, drugs that reverse the blockade must be readily available for emergency use. Anticholinesterases are used as reversal agents in this setting; however, the reversal agents are often used in conjunction with another agent (anti-muscarinic) to control side effects. The anticholinesterases inhibit the acetylcholinesterase enzyme from breaking down acetylcholine, thus contributing to the accumulation of ACh.

### Anticholinesterase Dosing for Localized Treatment

Given the localized nature of targeted administration (e.g., by injection), the effective dose would be at least an order of magnitude lower than either the oral or intravenous dosing. For example 0.1-2 mg delivered dose may be sufficient.

General guidance for conversion of the oral to IV dose, is to give patients 1/30^{th} of the oral dose. Since the targeted administration, is directly injected into the tissue, as compared to an IV administration, the dosing could be a ow as 0.1 mg or alternatively up to 1/10^{th} of the dose necessary for reversal of muscle relaxants.

In certain embodiments, the low dose is about ⅘ to about 1/50 of the clinical dose of the anticholinesterase when dosed for said anticholinesterase's oral or intravenous use (usually dosed for other therapeutic indication). In some embodiments, the low dose is about ⅕ to about 1/50 of the oral or intravenous clinical dosing, or about ⅕ to about 1/20, or about ⅕ to about 1/10, or about 1/10 to about 1/50, or about 1/20 to about 1/50, or about 1/10 of the oral or intravenous clinical dosing.

Dosing in topical approaches, without direct injection into the bladder muscle, will require a higher dose of medicament. Bioavailability will reduce the effective dose delivered to the bladder. For example, for a 0.1 mg desired dose, only 20% may be bioavailable, therefore the actual delivered dose would be 0.5 mg. The remaining 0.4 mg active agent will likely be lost due to washout.

Dose and volume of the instillation may vary. For example, in 20 mL volume for instillation, a dose of 100-150 mg/20 mL of pyridostigmine bromide may be used. Dose may vary depending on the details of formulation as discussed below.

In certain embodiments, the higher dose is about 5/4 to about 50/1 of the clinical dose of the anticholinesterase when dosed for said anticholinesterase's oral or intravenous use (usually dosed for other therapeutic indication). In some embodiments, the high dose is about 5/1 to about 50/1 of the oral or intravenous clinical dosing, or about 5/1 to about 20/1, or about 5/1 to about 10/1, or about 10/1 to about 50/1, or about 20/1 to about 50/1, or about 5/1 of the oral or intravenous clinical dosing.

In some embodiments of the disclosure, the anticholinesterase is administered in a dose of about 0.05-0.5 mg/kg, or in a dose of about 0.15-0.25 mg/kg, or in a dose of about 0.2 mg/kg. The specific dose of an anticholinesterase may be tailored to the individual based on the bladder surface area or volume (e.g., used an indirect estimate of relative surface area of the bladder to be treated), or any other dosing guidance provided for botulinum toxin injections known in the art. The specific dosage may also be tailored to the individual based on the size, with methods such as needle electromyographic guidance or nerve stimulation as an indicator of response. One of skill in the art will recognize that the dosages may be higher or lower, depending upon, among other factors, the activity of the anticholinesterase, the bioavailability of the anticholinesterase, its metabolism kinetics and other pharmacokinetic properties, the mode of administration and various other factors. One of skill in the art would also recognize that the anticholinesterase should be dosed in such manner not infiltrate or spread into systemic circulation such that it would cause unwanted side effects.

In some embodiments of the disclosure, the anticholinesterase may be administered immediately after the neurotoxin (e.g., botulinum toxin). For example, the anticholinesterase may be administered at least 1 minute, or at least 2 minutes, or at least 5 minutes, or at least 10 minutes, or at least 30 minutes after the neurotoxin. In some embodiments of the disclosure, the anticholinesterase may be administered sometime after the neurotoxin (e.g., botulinum toxin). For example, the anticholinesterase may be administered at least 1 hour, or at least 6 hours, or at least 24 hours, or at least 2 days, or at least 3 days, or at least 4 days, or at least 5 days, or at least 6 days, or at least 7 days after the neurotoxin.

As known in the art, the administration of the active agent to the bladder will be impacted by the rate of diffusion across the bladder surface, its concentration and temperature, in addition to the residence time, or time of exposure of the active agent in contact with the surface area of the bladder wall.

### Considerations for Clinical Use

The observation of urinary retention in a patient following botulinum toxin therapy is typically not diagnosed until one week post-therapy. According to clinical reports, the full onset of therapy is often subjective and can vary between 7 and 30 days. According to an evaluation of 35 patients, maximum improvement is not evidenced until an average of 8.3 days (range 2-20) (Rapp et al. (2007) International Braz J Urol 33(2):132-141), In normal practice, either the patient would notice the adverse effect of urinary retention and/or the urologist would make a diagnosis and subsequently, recommend rescue therapy.

In certain embodiments of the methods of the disclosure as described herein, the one or more anticholinesterases is administered by an injection (e.g., to the bladder wall or into the detrusor muscle).

In certain embodiments of the methods of the disclosure as described herein, the one or more anticholinesterases are administered by intravesical delivery. For example, the one or more anticholinesterases may be formulated in a manner similar to that currently used for intravesical delivery of BOTOX. Suitable examples include, but are not limited to, formulations used in current clinical trials.

In certain embodiments, the methods of the disclosure as described herein further comprises enhancing penetration of the administered the one or more anticholinesterase. Examples of medical devices or technologies which may be used to enhance drug penetration by disrupting the barrier membrane include, but are not limited to, shock waves or electromotive force.

### Targeted Administration

As noted above, the methods of the disclosure as described herein include administration of the one or more of anticholinesterases to a bladder muscle denervated by a botulinum toxin. Such administration is thus targeted to the site of action, and can enhance the one or more anticholinesterases' efficacy. Increasing local concentration of the one or more anticholinesterases to the afflicted tissue, in certain embodiments, minimizes the exposure to other areas of the body and thereby reduces toxicity. In certain embodiments, the targeted administration avoids hepatic first-pass metabolism and gastrointestinal tract side effects generally associated with anticholinesterases. In certain embodiments, the dose of the one or more anticholinesterases can be significantly lower. Lower dosage generally reduces the undesired systemic side effects. Thus, in certain embodiments, the methods of the disclosure allow for use of a variety of anticholinesterases that may or may not have serious or undesired side effects.

In certain embodiments of the methods of the disclosure as described herein, targeted administration of the one or more of anticholinesterases may be achieved by means of parenteral injection using conventional techniques, e.g., similar to those used in the intradetrusor injection of BOTOX.

In certain embodiments of the methods of the disclosure as described herein, targeted administration of the one or more of anticholinesterases may be achieved by means of topical administration by instillation (e.g., via catheter).

Such an approach, in certain embodiments, can be augmented to include one or more of transmucosal or mucoadhesive agents as described herein. Some examples of transmucosal and mucoadhesive agents include, but are not limited to, pectin and

In addition, the formulations suitable for topical administration by instillation can further include one or more penetration enhancers as described herein. In certain embodiments, the penetration enhancer is Poloxamer 407.

Transmucosal administration enables the anticholinesterase to enter the underlying tissue via the bladder wall surface area. As compared to an injection, intravesical instillation administration without injection will be less invasive, less prone to hematuria, and more time efficient. It should also be preferred by the patient, as it is less painful.

The performance (i.e., anticholinesterase uptake/transfer through the bladder wall) or bioavailability depends primarily on the anticholinesterase properties, such as molecule size, lipophilicity, polarity, and solubility. The anticholinesterase transfer may be further improved by longer exposure, and/or use of surfactants and/or penetration enhancers.

The methods of the disclosure are illustrated further by the following examples, which are not to be construed as limiting the invention in scope or spirit to the specific procedures described in them.

### EXAMPLES

### Example 1

### The Effect of Pyridostigmine Bromide on Muscle Force in the Detrusor Rat Muscle after Exposure to BOTOX®

A previously described in vitro model to measure bioactivity of botulinum neurotoxin type A in rat bladder muscle strips was used (van Uhm et al. (2014) BMC Urology, 14(37):1471-2490). The objective was to develop an assay to measure muscle performance in vitro when exposed to BOTOX and pyridostigmine (Pyr). After BOTOX administration, a drop-in force in a paralyzed detrusor muscle was expected. After exposure to Pyr, an increase in force would indicate recovery of the muscle. A modified bladder smooth muscle strip contractibility assay was used where was BOTOX administered by disrupting the bladder wall surface via 0.2 mm microneedle roller (Skinmedix, Naples, Fla.).

Preparation of Bladder Strips: The urinary bladder was isolated from an anesthetized rat and placed in warm Krebs-Henseleit buffer bubbled with 95% O₂/5% CO₂. The bladder was pinned at the dome and the base. The outer visceral peritoneum was carefully peeled off. An incision was then performed from base to dome and the bladder was flattened and pinned at its 4 corners. The urothelium was then carefully lifted from the muscle layer and closed scissor was inserted between the two layers. The jaws of the scissors were then opened while in the plane to create a small pocket. The process was repeated until the urothelium was separated, with occasional minimal cutting. Once the urothelium was removed, the dome and base of the bladder were cut longitudinally. Strips of about 1 mm by 8 mm were cut. At each end, tissue strips were attached to a bulldog tissue clip. Baseline Contractions: Muscle performance was measured in vitro with a 300B Aurora Scientific muscle lever system adapted with a horizontal perfusion bath. The bladder strip was placed in the horizontal bath and perfused with physiological buffer oxygenated with 95% O₂/5% CO₂ and kept at 25° C., The tissue clips were then mounted to a fixed post on one side, and a hook on the lever arm on the other. Each strip was then gently stretched until a baseline tension of 10 mN was achieved. The strip was left to equilibrate. Periodically, a brief 20 Hz, 0.5 s stimulation train was given and maximal tension noted. Baseline tension was adjusted until optimum length was achieved. The preparation was then left to equilibrate for one hour, or until baseline tension was stable and the spontaneous activity was regular. Electrical field stimulation (EFS) was then started (20 Hz, 0.2 ms pulse width, 5 second train, 5-minute interval) and force recorded for 100 minutes. Following equilibration and strip viability testing, the horizontal bath was drained, and the strips were detached from their hooks.

Treatment with BOTOX: BOTOX, 4 Units in 80 μL saline, was then applied, and a 0.2 mm microneedle roller was passed over the strips several times to ensure complete coverage of the strip. The strip was then allowed to incubate in the BOTOX for 2 hours. Penetration of BOTOX was enhanced by disruption of the bladder barrier surface by stripping of the urothelium layer (see FIG. 2), followed by microneedling. Disruption of skin surface is known to permit delivery of large molecules, such as botulinum toxins (about 150 kDa). Although pretreatment of the bladder wall surface is possible in an animal model, it is not practical to consider either microneedle pretreatment or a microneedle patch system in vivo into the bladder. For clinical drug delivery to the bladder, in a targeted administration, (1) intradetrusor injection or (2) intravesical delivery via instillation through a catheter as previously described would be preferred.

Care was taken to ensure that only minimal amount of buffer bathed the strip to prevent drying without diluting BOTOX. Following incubation, the bath was filled back with physiological buffer and the strips were reattached to the force transducer and electrical field stimulation re-started and force inhibition measured over 90 minutes. Control strips were incubated in saline without BOTOX to ensure strip viability throughout the assay.

Vehicle vs Treatment with Pyr: Following the 90-minute measurement, electrical field stimulation was suspended and either vehicle or 0.5 mM pyridostigmine was added to the bath. The strip was incubated for approximately 30 minutes. After 30 minutes, stimulation resumed, and the force was measured for 90 minutes. As a positive control, in one strip, following the measurement, 80 mM KCI was added to determine maximal force.

Results:

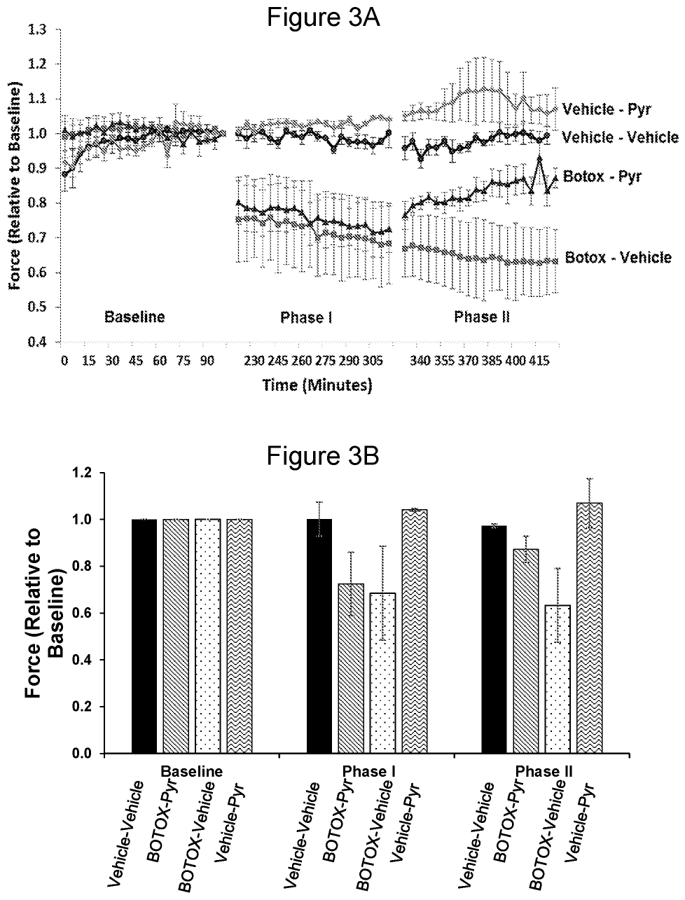

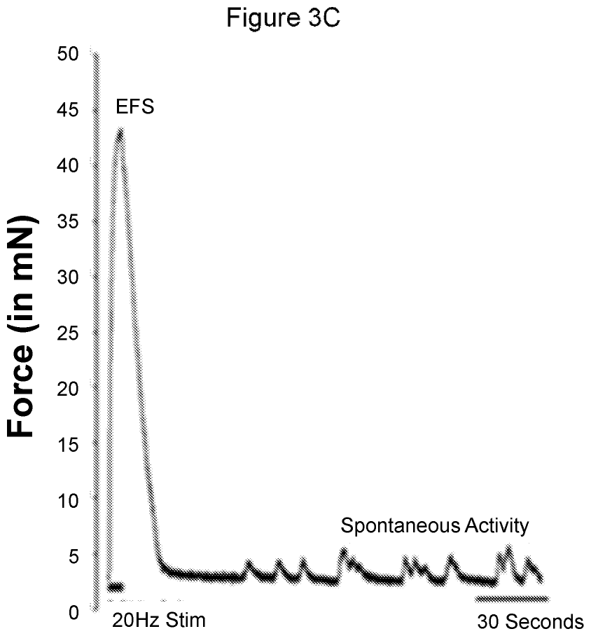

In contrast to the untreated bladder strip, BOTOX treated strips exhibited approximately 30% drop in force as illustrated in FIG. 3A. The drop-in force was comparable in both the vehicle and Pyr groups (28±14% drop vs. 32±20% drop for Pyr vs. vehicle, respectively). In the vehicle group, the drop-in force was stable until the end of the experiment (37±16% drop after 425 minutes). Exposure to pyridostigmine resulted in recovery of about 50% of the force drop (13±6% drop from baseline). The test and the vehicle group consisted of four or three muscle strips from juvenile female Sprague Dawley female rats, that weighed between 150-175 g, Juvenile rats were chosen because they exhibit spontaneous bladder contractions similar to OAB (Artim et al. (2011) Neurourol Urodyn, 30(8):1666-74).

### Example 2

### The Effect of Rivastigmine on Muscle Force in the Detrusor Rat Muscle after Exposure to BOTOX®

A previously described in vitro model to measure bioactivity of botulinum neurotoxin type A in rat bladder muscle strips was used (van Uhm et al. (2014) BMC Urology, 14(37):1471-2490). The objective is to develop an assay to measure muscle performance in vitro when exposed to BOTOX and rivastigmine (Riv). After BOTOX administration, a drop-in force in a paralyzed detrusor muscle was expected. After exposure to Rlv, an increase in force would indicate recovery of the muscle. A modified bladder smooth muscle strip contractibility assay is used as described in Example 1.

The bladder strips are prepared and muscle performance is measured as provided in Example 1.

Treatment with BOTOX: Treatment with BOTOX is performed as provided in Example 1. In short, 4 Units of BOTOX in 80 μL saline are applied, followed by passing of microneedle roller over the strips several times to ensure complete coverage of the strip. The strip is then allowed to incubated for 2 hours. Following incubation, the bath is filled back with physiological buffer and the strips are reattached to the force transducer and electrical field stimulation re-started and force inhibition is measured over 90 minutes, Control strips are incubated in saline without BOTOX.

Vehicle vs Treatment with Riv: Following the 90-minute measurement, electrical field stimulation is suspended and vehicle, 0.2 nM rivastigmine, 0.5 mM rivastigmine or 1 mM rivastigmine is added to the bath. The strip is incubated for approximately 30 minutes. After 30 minutes, stimulation is resumed, and the force is measured for 90 minutes. As a positive control, in one strip, following the measurement, 80 mM KCI is added to determine maximal force.

It is understood that the examples and embodiments described herein are for illustrative purposes only and that various modifications or changes in light thereof will be suggested to persons skilled in the art and are to be incorporated within the spirit and purview of this application and scope of the appended claims. All publications, patents, and patent applications cited herein are hereby incorporated herein by reference for all purposes.

What is claimed is:

1. A method of mitigating the side-effects of botulinum toxin therapy in a patient in need thereof, wherein the patient has received botulin toxin for treating overactive bladder or urinary urgency, the method comprising locally administering one or more of anticholinesterases to a bladder muscle denervated by a botulinum toxin.

2. The method of claim 1, wherein the botulinum toxin is botulinum toxin type A.

3. The method of claim 1, wherein the one or more anticholinesterases is selected from the group consisting of neostigmine, edrophonium, pyridostigmine, physostigmine, rivastigmine, donepezil, galantamine, and combinations thereof.

3. The method of claim 1, wherein the one or more anticholinesterases is selected from the group consisting of neostigmine, edrophonium, pyridostigmine, physostigmine, rivastigmine and combinations thereof.

4. The method claim 1, wherein the anticholinesterase is pyridostigmine.

5. The method claim 1, wherein the anticholinesterase is rivastigmine.

6. The method of claim 1, wherein the anticholinesterase is of formula: 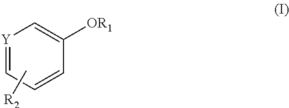
or a pharmaceutically acceptable salt thereof, wherein
Y is CR³ or N⁺X⁻R⁴, wherein X is a halogen;
R₁ is selected from hydrogen, C₁-C₆ alkyl, —CO(OH), —CO(C₁-C₆ alkoxy), —CO(NH₂), —CONH(C₁-C₆ alkyl), and —CON(C₁-C₆ alkyl)₂;
R₂ is hydrogen, or R₂ and R₃ together with the atoms to which they are attached form an optionally substituted heterocycle;
R₃ is selected from C₁-C₆ alkyl, C₁-C₆ alkoxy, hydroxy C₁-C₆ alkyl, amino C₁-C₆ alkyl, (C₁-C₆ alkylamino) C₁-C₆ alkyl, (di C₁-C₆ alkylamino) C₁-C₆ alkyl, C₁-C₆ alkoxy C₁-C₆ alkyl —OH, —NH₂, —NH(C₁-C₆ alkyl), —N(C₁-C₆ alkyl)₂, and —N⁴(C₁-C₆ alkyl)₃X⁻; and
R₄ is selected from C₁-C₆ alkyl, hydroxy C₁-C₆ alkyl, or C₁-C₆ alkoxy C₁-C₆ alkyl.

7. The method of claim 1, wherein the one or more anticholinesterases is a reversible anticholinesterase having one or more of groups selected from carbamate, tertiary ammonium, and quaternary ammonium,

8. The method of any of claims 1-7, wherein the locally administering is by intradetrusor injection into the bladder muscle.

9. The method of any of claims 1-7, wherein the locally administering is by intravesical instillation into the bladder or the bladder muscle.

10. The method of claim 9, wherein the intravesical instillation extends the dwell time of the one or more anticholinesterases within the bladder.

11. The method of claim 10, wherein the dwell time is extended by means of a medical device.

12. The method of claim 9, wherein the one or more anticholinesterase is formulated or co-administered with an agent to promote penetration into the bladder wall.

13. The method of claim 12, wherein the dwell time is extended by means of is formulating or co-administering with an agent.

14. The method of claim 13, wherein the agent is a mucoadhesive agent.

15. The method of claim 9, wherein the one or more of anticholinesterase is formulated into a pharmaceutical composition further comprising one or more mucoadhesives.

16. The method of claim 9, wherein the one or more of anticholinesterase is formulated into a pharmaceutical composition further comprising one or more penetration enhancers.

17. Use of one or more of anticholinesterases for mitigating the side-effects of botulinum toxin therapy in a patient in need thereof, wherein the patient has received botulin toxin for treating overactive bladder or urinary urgency, and wherein the use is by local administration to a bladder muscle denervated by a botulinum toxin.

18. The use of claim 17, wherein the botulinum toxin is botulinum toxin type A.

19. The use of claim 17, wherein the one or more anticholinesterases is selected from the group consisting of neostigmine, edrophonium, pyridostigmine, physostigmine, rivastigmine, donepezil, galantamine, and combinations thereof.

20. The use of claim 17, wherein the one or more anticholinesterases is selected from the group consisting of neostigmine, edrophonium, pyridostigmine, physostigmine_{;}
rivastigmine, and combinations thereof.

21. The use of claim 17, wherein the anticholinesterase is pyridostigmine.

22. The use of claim 17, wherein the anticholinesterase is rivastigmine.

23. The use of claim 17, wherein the anticholinesterase is of formula: 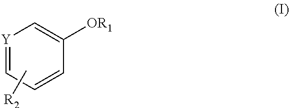
or a pharmaceutically acceptable salt thereof, wherein
Y is CR³ or N⁺X⁻R⁴, wherein X is a halogen;
R₁ is selected from hydrogen, C₁-C₆ alkyl, —CO(OH), —CO(C₁-C₆ alkoxy), —CO(NH₂), —CONH(C₁-C₆ alkyl), and —CON(C₁-C₆ alkyl)₂;
R₂ is hydrogen, or R₂ and R₃ together with the atoms to which they are attached form an optionally substituted heterocycle;
R₃ is selected from C₁-C₆ alkyl, C₁-C₆ alkoxy, hydroxy C₁-C₆ alkyl, amino C₁-C₆ alkyl, (C₁-C₆ alkylamino) C₁-C₆ alkyl, (di C₁-C₆ alkylamino) C₁-C₆ alkyl, C₁-C₆ alkoxy C₁-C₆ alkyl —OH, —NH₂, —NH(C₁-C₆ alkyl), —N(C₁-C₆ alkyl)₂, and —N⁴(C₁-C₆ alkyl)₃X⁻; and
R₄ is selected from C₁-C₆ alkyl, hydroxy C₁-C₆ alkyl, or C₁-C₆ alkoxy C₁-C₆ alkyl.

24. The use of claim 17, wherein the one or more anticholinesterases is a reversible anticholinesterase having one or more of groups selected from carbamate, tertiary ammonium, and quaternary ammonium.

25. The use of any of claims 17-24, wherein the locally administering is by intradetrusor injection into the bladder muscle.

26. The use of any of claims 17-24, wherein the locally administering is by intravesical instillation into the bladder or the bladder muscle.

27. The use of claim 26, wherein the intravesical instillation extends the dwell time of the one or more anticholinesterases within the bladder.

28. The use of claim 27, wherein the dwell time is extended by means of a medical device.

29. The use of claim 26, wherein the one or more anticholinesterase is formulated or co-administered with an agent to promote penetration into the bladder wall.

30. The use of claim 29, wherein the dwell time is extended by means of is formulating or co-administering with an agent.

31. The use of claim 30, wherein the agent is a mucoadhesive agent

32. The use of claim 26, wherein the one or more of anticholinesterase is formulated into a pharmaceutical composition further comprising one or more mucoadhesives.

33. The use of claim 26, wherein the one or more of anticholinesterase is formulated into a pharmaceutical composition further comprising one or more penetration enhancers.
